(19) United States
(12) Patent Application Publication
(10) Pub. No.: US 2026/0284250 A1
(43) Pub. Date: Sep. 24, 2026

(54) THERMALLY-MANAGED ELECTRONIC SCENTING CANDLE SIMULATOR WITH REALISTIC FRAGRANCE EMISSION

(71) Applicant: The Wild Worksmen, LLC dba Bianchi Candle Co., Papillion, NE (US)

(72) Inventors: Nicolas David Bianchi, Omaha, NE (US); Alan Tipp, Omaha, NE (US)

(73) Assignee: The Wild Worksmen, LLC dba Bianchi Candle Co., Papillion, NE (US)

(21) Appl. No.: 19/575,533

(22) Filed: Mar. 23, 2026

Related U.S. Application Data
(60) Provisional application No. 63/776,436, filed on Mar. 24, 2025.

(51) Int. Cl. A61L 9/14; F21S 6/00; F21S 9/02; F21S 10/04; F21Y 115/10

(52) U.S. Cl. CPC A61L 9/14; F21S 6/001; F21S 9/02; F21S 10/043; A61L 2209/12; A61L 2209/133; F21Y 2115/10

(57) ABSTRACT

Thermally managed nebulizing candle simulators having a light-and-aerosol sensory module and a sealed cartridge interface cooperate to provide realistic fragrance with light emission. The nebulizing candle simulator or electronic scenting candle includes a sensory module configured to emit light and fragrance, a nebulizing fragrance pump pneumatically coupled to the sensory module, and a cartridge interface fluidically couplable to the nebulizing fragrance pump and to a cartridge.

### CROSS REFERENCE TO RELATED APPLICATIONS

This application claims the benefit under 35 USC § 119(e) to U.S. Provisional Patent Application No. 63/776,436, filed Mar. 24, 2025, which is hereby incorporated by reference in its entirety.

### TECHNICAL FIELD

The present disclosure relates to electronic devices that simulate the sensory experience of a candle.

### BACKGROUND

Known electronic candles with fragrance features face several key shortcomings. Reed diffusion mechanisms, which are commonly found in wall plug-in devices, rely on heating elements to disperse fragrance through a reed or wick. These systems suffer from limited fragrance radius, requiring multiple units per room, and demonstrate poor efficiency in terms of fragrance utilization and dispersion. Additionally, the burn-through rate of fragrance in these systems provides only nominal effectiveness compared to the amount of fragrance consumed.

Water-based atomization systems, while more efficient than reed diffusion for dispersing fragrances, are limited by their requirement to use water as a carrier medium for essential oils. This constrains the types of fragrances that can be effectively dispersed. Furthermore, existing electronic fragrance devices often receive negative customer reviews due to issues like causing headaches, rapid fragrance depletion, and inconsistent fragrance output. The current market solutions fail to provide the seamless, realistic experience that consumers expect, particularly those who are sensitive to the quality and consistency of home fragrance products.

### BRIEF SUMMARY

The present disclosure provides a thermally managed nebulizing candle simulator in which a synchronized light-and-aerosol sensory module and a sealed cartridge interface cooperate to provide realistic fragrance with light emission.

According to an aspect, an electronic scenting candle comprises a sensory module comprising a simulated wick extending through a radial fragrance manifold, the simulated wick comprising one or more light emitting elements, wherein the radial fragrance manifold comprises a plurality of fragrance emission apertures extending around the simulated wick, the simulated wick being communicatively coupled with a controller. A nebulizing fragrance pump fluidically couples to the radial fragrance manifold, wherein the controller synchronizes illumination of the light emitting elements with operation of the nebulizing fragrance pump. A cartridge interface fluidically couples to the nebulizing fragrance pump and to a fragrance cartridge. An optional cosmetic shell encloses the nebulizing fragrance pump and the cartridge interface. An optional thermal conduction barrier is disposed between the cosmetic shell and the nebulizing fragrance pump.

According to another aspect, an electronic scenting candle comprises a sensory module comprising a simulated wick extending through a radial fragrance manifold, the simulated wick comprising one or more light emitting elements, wherein the radial fragrance manifold comprises a plurality of fragrance emission apertures extending around the simulated wick, the simulated wick being communicatively coupled with a controller. A nebulizing fragrance pump pneumatically couples to the radial fragrance manifold. A cartridge interface fluidically couples to the nebulizing fragrance pump and to a fragrance cartridge.

According to another aspect, an electronic scenting candle comprises a sensory module comprising a simulated wick extending through a radial fragrance manifold comprising a plurality of fragrance emission apertures extending around the simulated wick. A fragrance pump is pneumatically coupled to the radial fragrance manifold. A controller comprises a machine-readable non-transitory computer-readable medium and a processor storing logic, that when executed by the processor, synchronizes operation of the simulated wick and the fragrance pump. A cartridge interface fluidically couples to the fragrance pump and to a fragrance cartridge.

Embodiments of any of the foregoing aspects may comprise any one or more of the following features. The nebulizing fragrance pump may comprise a pump fluidically coupled to a nebulizer, a vacuum chamber, and the cartridge interface. The vacuum chamber may be fluidically disposed between the nebulizer and the pump. The sensory module further comprises a plenum coupled with the radial fragrance manifold. The thermal conduction barrier comprises a sleeve extending. The cosmetic shell comprises a wax material. The plurality of fragrance emission apertures extends through a simulated melt pool layer circumscribing the simulated wick. The cartridge interface comprises an inlet configured to form a fluid connection with a liquid contained within the fragrance cartridge when the fragrance cartridge engages a reversible engagement mechanism. The electronic scenting candle further comprises the fragrance cartridge. The controller comprises a machine-readable non-transitory computer-readable medium and a processor storing logic, that when executed by the processor, synchronizes operation of the simulated wick and the nebulizing fragrance pump. The controller synchronizes illumination from the simulated wick with activation of the nebulizing fragrance pump. The plurality of fragrance emission apertures extends through a simulated melt pool layer circumscribing the simulated wick. The nebulizing fragrance pump comprises a pump pneumatically coupled to a nebulizer, a vacuum chamber, and the cartridge interface. The cartridge interface comprises an inlet configured to form a fluid connection with a liquid contained within the fragrance cartridge when the cartridge interface engages a reversible engagement mechanism of the cartridge interface. The cartridge interface is a modular unit configured to slidably decouple from the nebulizing fragrance pump. The candle further comprises the fragrance cartridge, wherein the fragrance cartridge is configured to reversibly lock into the cartridge interface. The candle further comprises a thermal conduction barrier disposed between a cosmetic shell and a harness supporting the nebulizing fragrance pump and the cartridge interface. The sensory module comprises a plurality of light emitting diodes disposed on a printed circuit board and positioned to emit light through the cosmetic shell.

### BRIEF DESCRIPTION OF THE DRAWINGS

Reference is now made to the following descriptions taken in conjunction with the accompanying figures and the appendix, in which:

FIG. 1 is a first perspective view of an electronic scenting candle of the present disclosure.

FIG. 2 is a second perspective view of the electronic scenting candle of the present disclosure.

FIG. 3 is an exploded view of the electronic scenting candle of FIG. 1.

FIG. 4 is a first side section view of the electronic scenting candle of FIG. 1

FIG. 5 is a perspective partial exploded section view of the electronic scenting candle of FIG. 1.

FIG. 6 is a perspective view of a sensory module of the electronic scenting candle of FIG. 1.

### DETAILED DESCRIPTION

The present disclosure provides electronic scenting candles and compatible fragrance cartridges that realistically simulate the light and fragrance of a real candle.

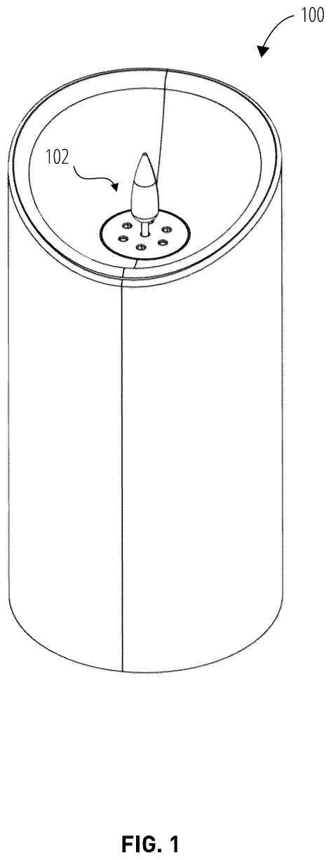

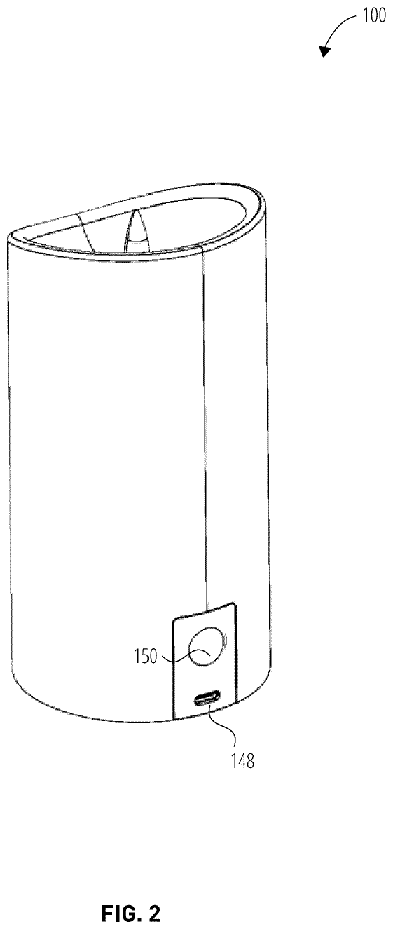

FIG. 1 and FIG. 2 show perspective views of an electronic scenting candle 100 according to the present disclosure that mimics the appearance of a pillar candle and is compatible with a removable fragrance cartridge. Outwardly visible aspects of the electronic scenting candle 100 include a cosmetic shell 118 enclosing light and fragrance-emitting elements as described below, a sensory module 102 that simulates the luminosity and scent of a candle, a user interface 150 for toggling through different modes, and a charging interface 148 for the internal battery.

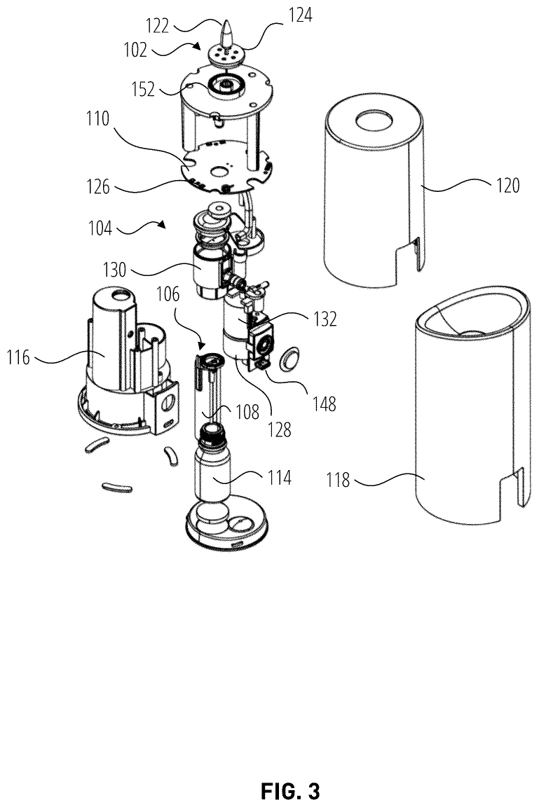

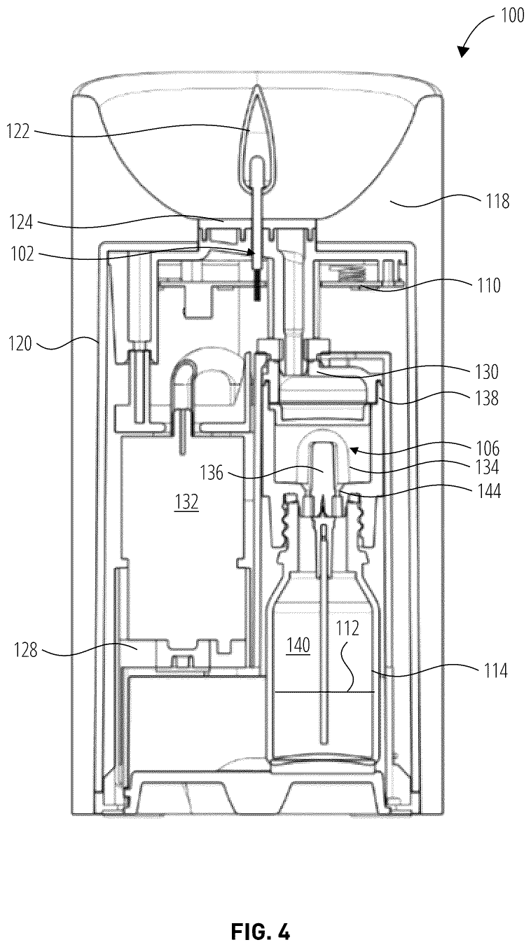

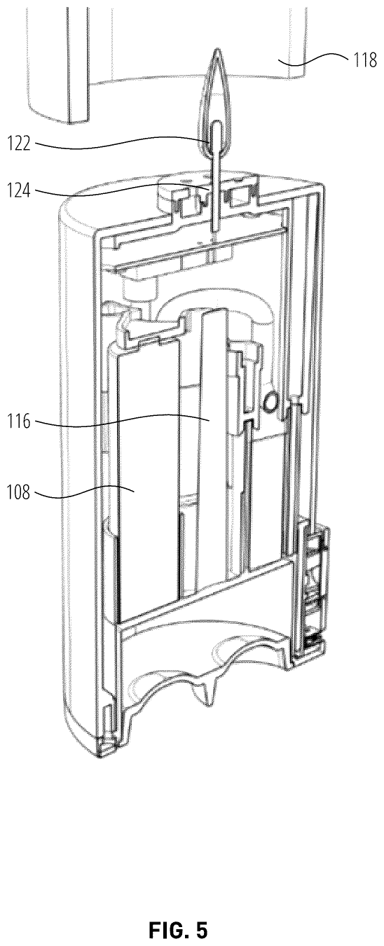

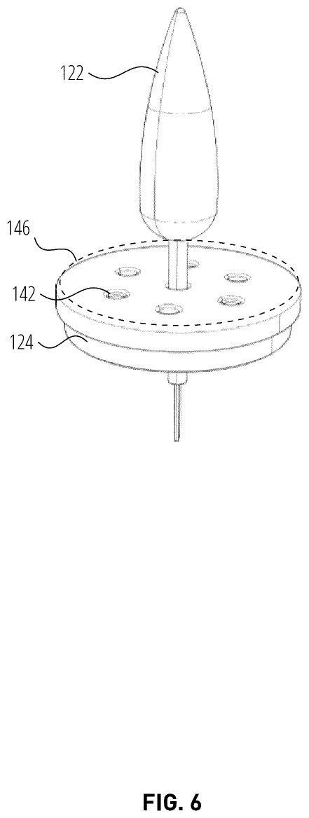

FIG. 3 shows the electronic scenting candle 100 as an exploded assembly to reveal various aspects thereof. FIG. 4 shows a first section view of the electronic scenting candle 100, whereas FIG. 5 shows a second section view thereof. FIG. 6 shows aspects of the sensory module 102. FIG. 3-FIG. 6 will now be described together.

The electronic scenting candle 100 includes a sensory module 102 configured to emit light and aerosol fragrance, a nebulizing fragrance pump 104 pneumatically coupled to the sensory module 102, and a cartridge interface 106 fluidically couplable to the nebulizing fragrance pump 104. The sensory module 102 and nebulizing fragrance pump 104 are operably connected to a battery 108, e.g., a rechargeable Li-ion battery and a controller 110 that are in electrical communication with the charging interface 148. Further, the sensory module 102 is fluidically connected to the nebulizing fragrance pump 104 that draws liquid fragrance 112 from a fragrance cartridge 114, nebulizes the liquid fragrance into a fine aerosol, and emits the aerosol fragrance through the sensory module 102 into the environment surrounding the electronic scenting candle 100.

Aspects of the sensory module 102, nebulizing fragrance pump 104, and cartridge interface 106 are contained within or supported by a harness 116 that is disposed within the cosmetic shell 118. To prevent the cosmetic shell 118 from melting from heat created by the electronics, the electronic scenting candle 100 includes a thermal conduction barrier 120 formed as a sleeve of insulating fiber or polymer having a relatively low thermal conductivity and extending around an internal circumference of the cosmetic shell 118. In some embodiments, the thermal conduction barrier 120 is disposed between the cosmetic shell 118 and the harness 116 and formed.

In some embodiments, the cosmetic shell 118 is formed of a polymeric wax analog or a wax material such as paraffin, soy wax, beeswax, palm wax, microcrystalline wax, gel wax, or blends thereof, optionally with pigments, opacifiers, fragrance-neutral fillers, or surface modifiers selected to emulate the look and feel of a poured candle. The shell 118 may have a thickness from about 1 mm to about 30 mm, and in certain embodiments may include a translucency gradient in which an upper region adjacent the sensory module 102 is more translucent than a lower region so that light from the sensory module 102 appears to glow through the shell in a candle-like manner while the lower region remains more opaque. In some embodiments, the cosmetic shell 118 includes an outer wax or wax-analog layer and an inner structural liner or sleeve so that realism and dimensional stability are both promoted.

In some embodiments, the thermal conduction barrier 120 comprises an insulating sleeve formed as a layer or a multi-layer stack including one or more air gaps, standoffs, reflective films, low-thermal-conductivity polymers, silicone foams, polyimide films, ceramic papers, mica sheets, aerogel blankets, fiberglass mats, basalt fiber mats, aramid fiber mats, cork layers, nonwoven fibers, or combinations thereof. The thermal conduction barrier 120 may have an overall thickness from about 0.25 mm to about 10 mm and an effective thermal conductivity of less than about 0.25 W/m-K, less than about 0.15 W/m-K, or less than about 0.08 W/m-K. The thermal conduction barrier 120 cooperates with the harness 116 to maintain an outer surface temperature of the cosmetic shell 118 below a wax-softening or deformation temperature threshold during sustained operation, while allowing the cosmetic shell 118 to retain a realistic candle profile.

In some embodiments, hidden structural and fluid-routing features are used so that ordinary viewing angles present the appearance of a conventional candle rather than an exposed diffuser. By way of example, fluid paths may be routed through interior channels of the harness 116, within an annular plenum 152 disposed below the radial fragrance manifold 124, or through concealed passages beneath an upper melt-pool region (simulated melt pool layer 146). Likewise, cartridge access may be provided through a lower door, underside opening, or base cap in a bottom end of the electronic scenting candle 100 (i.e., an opposite end from the simulated wick 122), said opening being optionally masked by a cap, door, or other covering. Internal supports for the battery 108, fragrance pump 128, and cartridge interface 106 such as harness 116 may be positioned radially inward so that the silhouette of the electronic scenting candle 100 remains candle-like from typical user viewpoints.

The sensory module 102 enhances visual and fragrance realism and comprises an electronic simulated wick 122 extending through a radial fragrance manifold 124 that is pneumatically coupled to the nebulizing fragrance pump 104 via the plenum 152. The simulated wick 122 includes one or more light emitting elements (e.g., light emitting diodes) disposed on a wick or mast that emit light at a wavelength and frequency to simulate a burning wick. In some embodiments, the sensory module 102 further comprises the controller 110, which includes a machine-readable non-transitory computer-readable medium and a processor storing logic, that when executed by the processor, control operation of the simulated wick 122 and the nebulizing fragrance pump 104 in order to increase realism. The controller 110 may be at least partially embodied on one or more printed circuit board (PCBs). In some embodiments, the controller 110 synchronizes illumination from the simulated wick 122 with activation of the nebulizing fragrance pump 104 such that both activate simultaneously. In some embodiments, the sensory module 102 comprises a plurality of light emitting element 126 positioned around the PCB to emit light through the cosmetic shell 118 for added realism.

In some embodiments, the simulated wick 122 includes multiple light-emitting regions at different heights, colors, or diffusion characteristics to emulate a candle flame, ember, and wick stem. For example, the sensory module 102 may include amber, red, yellow, and/or white light-emitting diodes arranged within or adjacent the simulated wick 122, together with a light pipe, reflective collar, frosted sleeve, translucent wick body, scattering particles, or other optical diffusion structures that obscure direct point-source visibility and produce a softer flame-like luminance distribution. Such optical structures may also be shaped to preserve the appearance of a centered wick when viewed from different directions.

In some embodiments, the controller 110 stores and executes one or more candle-specific synchronization profiles that couple light emission of the simulated wick 122 and fragrance emission from the nebulizing fragrance pump 104. For example, a startup sequence may imitate a candle catching by first driving the simulated wick 122 at a relatively low amber intensity, then ramping intensity and flicker amplitude over a period of about 0.2 seconds to about 5 seconds, and thereafter initiating a low-duty-cycle activation of the nebulizing fragrance pump 104 so that the onset of fragrance follows the apparent ignition of the flame. A warm mode may progressively increase fragrance pulse width or pulse frequency. A steady mode may maintain quasi-random flicker with intermittent fragrance pulses, and an extinguish mode may reduce light output and fragrance output together or in an offset sequence that mimics snuffing of a real candle. Such modes may optionally be toggled by the user via the user interface 150.

Turning to FIG. 3-FIG. 5, the nebulizing fragrance pump 104 comprises a fragrance pump 128 pneumatically coupled to an ultrasonic aerosol creation mechanism, i.e., a nebulizer 130, a vacuum chamber 132, and the cartridge interface 106.

In some embodiments, the vacuum chamber 132 functions as a buffer volume that smooths transient pressure variations produced by the fragrance pump 128, separates liquid transport from instantaneous pump pulsation, and stabilizes liquid fragrance feed to the nebulizer 130. The vacuum chamber 132 may be positioned upstream of the nebulizer 130, downstream of the fragrance pump 128, integrated with the cartridge interface 106, or divided into multiple sub-chambers. By damping rapid pressure excursions, the vacuum chamber 132 can reduce spitting, large-droplet ejection, startup surges, and end-of-cartridge sputter, thereby helping the fragrance plume remain fine and visually unobtrusive while also improving consistency of scent output over time.

In some embodiments, the liquid fragrance 112 is a substantially waterless formulation having a viscosity at 25 degrees C from about 1 centipoise to about 250 centipoise, for example from about 3 centipoise to about 100 centipoise, and may include one or more essential oils, aroma chemicals, perfume oils, carrier oils, stabilizers, antioxidants, low-volatility solvents, or combinations thereof. The nebulizing fragrance pump 104 may be configured to generate droplets having a volume median diameter from about 0.5 micrometers to about 15 micrometers, for example from about 1 micrometer to about 8 micrometers, at a fragrance emission rate from about 0.1 mg/min to about 20 mg/min, with a room-scale plume radius from about 0.3 m to about 5 m depending on selected mode. In some embodiments, low-power operation is used during ambient candle-like use so that fragrance dispersal remains effective without creating a humidifier-like visible cloud.

In some embodiments, acoustic and fluidic tuning features are employed so that the device behaves more like a candle than a conventional diffuser. By way of example, the fragrance pump 128 and/or nebulizer 130 may be mounted on elastomeric grommets, resilient pads, floating brackets, or other isolation mounts that reduce vibration transfer into the harness 116 and cosmetic shell 118. Fluid paths may include mufflers, labyrinth passages, expansion cavities, porous dampers, or acoustic foam to reduce tonal noise, and the fluid path between the cartridge interface 106 and the nebulizer 130 may be shortened, smoothed, and configured with minimal dead volume to reduce retained liquid and stale fragrance. Filters, capillary restrictors, anti-clogging meshes, purge cycles, reverse-pulse clearing cycles, and/or dry-air blow-off cycles may also be used to reduce fouling of the nebulizing fragrance pump 104, especially when higher-viscosity fragrance oils are used.

Referring to FIG. 4 and FIG. 5, the cartridge interface 106 enables secure insertion and removal of the fragrance cartridge 114, and comprises an inlet 134 configured to form a fluid connection through a nozzle 136 with liquid contained within the fragrance cartridge 114 when it engages a reversible engagement mechanism 138 of the cartridge interface 106. In some embodiments, the cartridge interface 106 comprises at least one of a radial seal or a locking actuator 144. Representative locking actuators include mechanically-biased (e.g., spring-loaded) push-push mechanisms, e.g., pop lock mechanisms. In some embodiments, the cartridge interface 106 is a modular unit configured to slidably decouple from the nebulizing fragrance pump 104, e.g., for periodic replacement.

In some embodiments, the cartridge interface 106 is a cartridge interface 106 configured so that a single insertion motion both positions and locks the fragrance cartridge 114. More particularly, the fragrance cartridge 114 may travel along an insertion axis until a final portion of travel causes the nozzle 136 and inlet 134 to establish a sealed fluid path, whereupon the reversible engagement mechanism 138 automatically latches to retain the fragrance cartridge 114 against withdrawal. A subsequent push, button actuation, slide actuation, or other release input may de-latch the reversible engagement mechanism 138 and permit partial spring-assisted or resilient ejection of the cartridge 114 so that the user can grasp and remove it. Such a push-push sequence allows replacement of the cartridge 114 without requiring twisting of the candle body or access to internal tubing.

In some embodiments, the fragrance cartridge 114 and cartridge interface 106 cooperate to provide self-sealing fluidic engagement. The nozzle 136 may be embodied as a blunt cannula, tapered spigot, hollow spike, or valve-opening stem, while the cartridge 114 may include a septum, slit valve, duckbill valve, umbrella valve, ball check, spring-biased poppet, or other biased closed structure that opens after insertion reaches a predetermined position. Redundant seals may be used, including one or more radial seals, axial face seals, O-rings, lip seals, or gasketed interfaces, so that air ingress and liquid leakage are reduced both during operation and during cartridge replacement. Anti-leak, anti-drip, anti-backflow, siphon-break, capillary-trap, absorbent-capture, and check-valve structures may also be included within the fluid pathways between cartridge interface 106 and the nebulizer 130 to the so that the cartridge 114 can be replaced with minimal user exposure to liquid fragrance and, in some embodiments, without leakage even if the electronic scenting candle 100 is tilted or inverted during handling.

In some embodiments, keyed insertion features on ensure proper cartridge orientation and inhibit incorrect installation. For example, the cartridge 114 and cartridge interface 106 may include keyed insertion features such as complementary shaped surfaces, asymmetric tabs and slots, unequal rails, offset guide ribs, chamfers, magnets, coded detents, or other polarization features that accept a particular rotational orientation before full insertion is permitted. The user may receive tactile and/or audible feedback, such as a detent rise, click, or stop, to confirm a fully seated condition. In some embodiments, the locked condition corresponds to simultaneous mechanical retention, valve opening, and fluid sealing.

In some embodiments, the electronic scenting candle 100 further comprises the fragrance cartridge configured to reversibly pop lock into the cartridge interface 106, e.g., with sliding actuation. The fragrance cartridge 114 includes a fragrance reservoir 140 containing a liquid fragrance, e.g., an oil-based formulation. The reservoir 140 may have different shapes in different embodiments, for example a cylindrical shape or an irregular shape such as a gooseneck, e.g., to increase volume of the reservoir 140.

In some embodiments, the fragrance cartridge 114 is itself a separately manufacturable and claimable article configured for use with the electronic scenting candle 100. The cartridge 114 may have an annular, partially annular, crescent, horseshoe, shaped, multi-lobed, cylindrical, or gooseneck geometry selected to wrap around one or more internal components of the candle while preserving an external candle silhouette and maximizing reservoir volume. The reservoir 140 may include one or more internal baffles, capillary structures, weighted dip tubes, flexible dip tubes, or low-point pickups so that fragrance remains accessible across a range of orientations and fill levels.

In some embodiments, the cartridge 114 includes headspace-control and venting features such as a hydrophobic membrane vent, one-way vent, collapsible bag, piston, rolling diaphragm, or other structure that allows liquid withdrawal while limiting oxidation, leakage, or pressure imbalance. The cartridge 114 may carry a single fragrance or multiple isolated fragrances in separate compartments that can be selectively communicated to the cartridge interface 106 through a selector valve, rotatable port, rupturable seal, or electronically commanded valve. Disposable, refillable, and recyclable cartridge formats are contemplated, as are child-resistant and tamper-evident features such as push-and-slide releases, tear bands, breakaway tabs, or locked refill caps. In some embodiments, the cartridge 114 additionally includes an identification element such as an RFID tag, NFC tag, EEPROM, resistor code, color code, barcode, or other memory/coding feature that identifies fragrance type, authenticity, remaining life, recommended control settings, and/or safety information to the controller 110.

Referring to FIG. 6, the radial fragrance manifold 124 comprises a plurality of fragrance emission apertures 142 extending around the simulated wick 122. Advantageously, this arrangement directs fragrance aerosol toward the simulated wick 122 such that during use, the electronic scenting candle 100 conveys the impression that the fragrance is being emitted from the simulated wick 122. Different embodiments of the radial fragrance manifold 124 may have different shapes, numbers, and radial arrangements of the fragrance emission apertures 142 that direct fragrance aerosol toward the 122. In some embodiments, a transparent/translucent polymer layer 146 is disposed upon and/or around the radial fragrance manifold 124 and around the simulated wick 122 to impart a realistic visual impression of melted wax. In some such embodiments, the fragrance emission apertures 142 extend through the transparent/translucent polymer layer, further enhancing the realism of the electronic scenting candle 100.

In some embodiments, the radial fragrance manifold 124 is annular, partially annular, polygonal, lobed, scalloped, multi-segmented, or defined by arcuate sections disposed around the simulated wick 122. The fragrance emission apertures 142 may form an array numbered from about 2 to about 60, for example from about 6 to about 24, and may be circular, oval, slotted, teardrop-shaped, crescent-shaped, or formed as annular segments. Each aperture 142 may have a diameter from about 0.1 mm to about 1.5 mm, and adjacent apertures may be equally spaced around 360 degrees, concentrated within selected arcs, arranged in concentric rings, offset rings, or asymmetric groupings, or otherwise tuned to shape the fragrance plume in a desired manner.

In some embodiments, an inner edge of the radial fragrance manifold 124 is spaced from the simulated wick 122 by about 0.2 mm to about 10 mm, and the upper outlet plane of the apertures 142 may be flush with, recessed below a surrounding top surface by about 0 mm to about 5 mm. The simulated wick 122 may project above the manifold 124 by about 1 mm to about 25 mm. Aperture axes may be directed vertically, upwardly and inwardly toward the wick, upwardly and tangentially to induce local swirl, upwardly and outwardly to broaden ambient diffusion, or in mixed angular patterns. In this manner, a substantial portion of the fragrance aerosol can pass through an illuminated zone adjacent the simulated wick 122, causing the emitted fragrance to be visually associated with the flame effect rather than with a discrete side vent or visible diffuser outlet.

In some embodiments, the simulated melt pool layer 146 is formed from a silicone or other material as an annular ring, meniscus, pooled melt-wax analog, irregular drip field, or other top-surface feature around the simulated wick 122. The layer 146 may partially cover, surround, or extend through the apertures 142 so as to conceal hard edges of the manifold 124, alter plume breakup, diffuse or spread individual jets, and reduce direct visibility of emission points. In certain embodiments, fragrance exits through a ring around the simulated wick 122. In other embodiments, at least a majority, such as at least 50%, at least 70%, or at least 90% of total fragrance output exits through apertures disposed around the simulated wick 122, with any remainder being discharged through optional hidden auxiliary outlets to tune room-scale dispersion while preserving the perceptual impression that scent originates from the flame/wick region.

In some embodiments, the simulated melt pool layer 146 further defines a melt-pool simulation including a concave well, annular moat, terraced ledge, sloped meniscus, or irregular poured-wax contour surrounding the simulated wick 122. The fragrance manifold 124, polymer layer 146, and any auxiliary optical components may be recessed within or blended into this top-surface geometry so that the user perceives a realistic melted-wax appearance rather than a distinct diffuser assembly. This top-surface configuration can cooperate with the optical structures of the sensory module 102 to preserve a realistic silhouette and flame presentation from ordinary viewing angles.

In some embodiments, the foregoing structural relationships produce technical effects that are measurable in addition to being visually pleasing. For example, the vacuum chamber 132, tuned aperture geometry of the radial fragrance manifold 124, and synchronized control of the nebulizing fragrance pump 104 can yield a lower visible plume than humidifier-style atomizers, a more consistent fragrance output over time, and reduced fragrance wastage associated with continuous passive diffusion. Likewise, the thermal conduction barrier 120, hidden airflow architecture, and vibration-isolated mounting of the fragrance pump 128 can contribute to lower shell temperatures and quieter operation. In some embodiments, the electronic scenting candle 100 may be configured such that shell temperature, fragrance-output variation, and acoustic noise at a selected distance fall within predetermined thresholds that support candle-like use in bedrooms, living spaces, hospitality settings, or other scent-sensitive environments.

In some embodiments, user-panel or consumer testing may show increased perceived realism when compared with devices in which fragrance exits from a side vent, from a visibly humidified plume, or independently of flame-simulation timing. Without wishing to be bound by theory, the wick-centered emission geometry, concealed fluid paths, melt-pool analog, and temporal coupling of light and fragrance can cause users to attribute scent emission to the apparent flame location, thereby improving realism while maintaining efficient fragrance delivery.

The foregoing features, taken alone or in any combination, enhance the realism of the electronic scenting candle 100. For example, the nebulizer 130 creates a fine aerosol from the liquid fragrance 112 which is barely perceptible when emitted through the sensory module 102 and which has a wide emission radius. Such characteristics are not possible with other scent-creation mechanisms, for example reed diffusion mechanisms or water-based atomizers. As another example, the cosmetic shell 118 realistically simulates the look and feel of a real candle. A cosmetic shell 118 is possible in the electronic scenting candle 100 by the thermal conduction barrier 120 that prevents it from melting. As still another example, functions of the electronic scenting candle 100 may be synchronized by the controller 110, namely the activation of the nebulizer 130 and simulated wick 122, such that the electronic scenting candle 100 emits light simultaneously or contemporaneously with the emission of fragrance aerosol. The realistic flame effect from the simulated wick 122 further enhances realism.

Various changes can be made to the embodiments of the present disclosure as could be reasonably contemplated in view of the above-described description by any person skilled in the art. The following claims are presented as examples of embodiments of the present disclosure, but these claims should not be construed to limit other claims or other embodiments disclosed herein.

The detailed description set forth above in connection with the appended drawings, where like numerals reference like elements, are intended as a description of representative embodiments of the present disclosure and are not intended to represent the only embodiments. Each embodiment described m this disclosure is provided as an example or illustration and should not be construed as preferred or advantageous over other embodiments. The illustrative embodiments provided herein are not intended to be exhaustive or to limit the disclosure to the precise forms disclosed. Similarly, any steps described herein may be interchangeable with other steps, or combinations of steps, in order to achieve the same or substantially similar result. Further still, one or more features of any embodiment may be combined with one or more features of one or more embodiments to form additional embodiments, which are within the scope of the present disclosure.

Generally, the embodiments disclosed herein are non-limiting, and the inventors contemplate that other embodiments within the scope of this disclosure may include structures and functionalities from more than one specific embodiment shown in the FIGURES and described in the specification. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed. For example, the present disclosure includes additional embodiments having combinations of any one or more features described above with respect to the representative embodiments.

In the foregoing description, specific details are set forth to provide a thorough understanding of representative embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure.

The present application may include references to directions, such as “first,” “second,” “vertical,” “horizontal,” “front,” “rear,” “left,” “right,” “top,” and “bottom,” “below,” “around,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” means any number that is more than one, for example, two, three, four, five, etc. The term “about,” “approximately,” etc., means plus or minus 5% of the stated value. The term “based upon” means “based at least partially upon.” The term “between” includes the values recited in connection therewith. The expressions “at least one of A, B, or C”; “at least one of A, B, and C”; and “at least one of A, B, and/or C” have the same meaning, i.e., any one of the following conditions satisfy all of the foregoing expressions: A; B; C; AB; AC; BC; ABC.

### LISTING OF DRAWING ELEMENTS

100 electronic scenting candle
102 sensory module
104 nebulizing fragrance pump
106 cartridge interface
108 battery
110 controller
112 fragrance
114 fragrance cartridge
116 harness
118 cosmetic shell
120 thermal conduction barrier
122 simulated wick
124 radial fragrance manifold
126 light emitting element
128 fragrance pump
130 nebulizer
132 vacuum chamber
134 inlet
136 nozzle
138 reversible engagement mechanism
140 reservoir
142 fragrance emission apertures
144 locking actuator
146 simulated melt pool layer
148 charging interface
150 user interface
152 plenum

What is claimed is:

1. An electronic scenting candle, comprising:
a sensory module comprising a simulated wick extending through a radial fragrance manifold, the simulated wick comprising one or more light emitting elements, wherein the radial fragrance manifold comprises a plurality of fragrance emission apertures extending around the simulated wick, the simulated wick being communicatively coupled with a controller;
a nebulizing fragrance pump pneumatically coupled to the radial fragrance manifold, wherein the controller synchronizes illumination of the light emitting elements with operation of the nebulizing fragrance pump;
a cartridge interface fluidically couplable to the nebulizing fragrance pump and to a fragrance cartridge;
a cosmetic shell enclosing the nebulizing fragrance pump and the cartridge interface; and
a thermal conduction barrier disposed between the cosmetic shell and the nebulizing fragrance pump.

2. The electronic scenting candle of claim 1, wherein the nebulizing fragrance pump comprises a pump fluidically coupled to a nebulizer, a vacuum chamber, and the cartridge interface.

3. The electronic scenting candle of claim 2, wherein the vacuum chamber is fluidically disposed between the nebulizer and the pump.

4. The electronic scenting candle of claim 2, wherein the sensory module further comprises a plenum coupled with the radial fragrance manifold.

5. The electronic scenting candle of claim 2, wherein the thermal conduction barrier comprises a sleeve extending around an internal circumference of the cosmetic shell.

6. The electronic scenting candle of claim 2, wherein the cosmetic shell comprises a wax material.

7. The electronic scenting candle of claim 1, wherein the plurality of fragrance emission apertures extends through a simulated melt pool layer circumscribing the simulated wick.

8. The electronic scenting candle of claim 1, wherein the cartridge interface comprises an inlet configured to form a fluid connection with a liquid contained within the fragrance cartridge when the fragrance cartridge engages a reversible engagement mechanism.

9. The electronic scenting candle of claim 1, further comprising the fragrance cartridge.

10. An electronic scenting candle, comprising:
a sensory module comprising a simulated wick extending through a radial fragrance manifold, the simulated wick comprising one or more light emitting elements, wherein the radial fragrance manifold comprises a plurality of fragrance emission apertures extending around the simulated wick, the simulated wick being communicatively coupled with a controller;
a nebulizing fragrance pump pneumatically coupled to the radial fragrance manifold; and
a cartridge interface fluidically couplable to the nebulizing fragrance pump and to a fragrance cartridge.

11. The electronic scenting candle of claim 10, wherein the controller comprises a machine-readable non-transitory computer-readable medium and a processor storing logic, that when executed by the processor, synchronizes operation of the simulated wick and the nebulizing fragrance pump.

12. The electronic scenting candle of claim 11, wherein the controller synchronizes illumination from the simulated wick with activation of the nebulizing fragrance pump.

13. The electronic scenting candle of claim 10, wherein the plurality of fragrance emission apertures extends through a simulated melt pool layer circumscribing the simulated wick.

14. The electronic scenting candle of claim 10, wherein the nebulizing fragrance pump comprises a pump pneumatically coupled to a nebulizer, a vacuum chamber, and the cartridge interface.

15. The electronic scenting candle of claim 10, wherein the cartridge interface comprises an inlet configured to form a fluid connection with a liquid contained within the fragrance cartridge when the cartridge interface engages a reversible engagement mechanism of the cartridge interface.

16. The electronic scenting candle of claim 10, wherein the cartridge interface is a modular unit configured to slidably decouple from the nebulizing fragrance pump.

17. The electronic scenting candle of claim 10, further comprising the fragrance cartridge, wherein the fragrance cartridge is configured to reversibly lock into the cartridge interface.

18. The electronic scenting candle of claim 10, further comprising a thermal conduction barrier disposed between a cosmetic shell and a harness supporting the nebulizing fragrance pump and the cartridge interface.

19. The electronic scenting candle of claim 18, wherein the sensory module comprises a plurality of light emitting diodes disposed on a printed circuit board and positioned to emit light through the cosmetic shell.

20. An electronic scenting candle, comprising:
a sensory module comprising a simulated wick extending through a radial fragrance manifold comprising a plurality of fragrance emission apertures extending around the simulated wick;
a fragrance pump pneumatically coupled to the radial fragrance manifold;
a controller comprising a machine-readable non-transitory computer-readable medium and a processor storing logic, that when executed by the processor, synchronizes operation of the simulated wick and the fragrance pump; and
a cartridge interface fluidically couplable to the fragrance pump and to a fragrance cartridge.
